(19) United States
(12) Patent Application Publication
(10) Pub. No.: US 2022/0346670 A1
(43) Pub. Date: Nov. 3, 2022

(54) METHOD FOR DETECTING GAIT EVENTS BASED ON ACCELERATION

(71) Applicant: Wuhan Qiwu Technology Co., Ltd., Wuhan (CN)

(72) Inventors: Kun CHEN, Wuhan (CN); Xiaoqing YANG, Wuhan (CN); Yuyin GENG, Wuhan (CN)

(21) Appl. No.: 17/378,813

(22) Filed: Jul. 19, 2021

(30) Foreign Application Priority Data
Apr. 30, 2021 (CN) 2021104887993

(51) Int. Cl. A61B 5/11; A61B 5/00

(52) U.S. Cl. CPC A61B 5/112; A61B 5/6801; A61B 5/725; A61B 5/7282; A61B 2562/0219

(57) ABSTRACT

A method for detecting gait events based on acceleration, comprising the following steps: obtaining a three-axis acceleration energy signal and a three-axis acceleration signal, and filtering and smoothing the three-axis acceleration energy signal and the three-axis acceleration signal; using a peak detection and a zero-crossing detection to screen points of local maximum and exceeding the peak threshold from processed vertical acceleration signal to form a point set ZC2; for any point ZCₖ in the point set ZC2, searching for the maximum value of the AP acceleration within a preset search window centered on the abscissa of the point ZCₖ. By only analyzing three-axis acceleration data for gait event recognition, the utilization of hardware resources is improved, the number of sensors is reduced, the accuracy of gait event detection is improved, and the consumption of computing resources is reduced.

### CROSS-REFERENCE TO RELATED APPLICATIONS

The application claims priority to Chinese patent application No. 202110488799.3, filed on Apr. 30, 2021, the entire contents of which are incorporated herein by reference.

### TECHNICAL FIELD

The application relates to the technical field of pattern recognition, in particular to a method for detecting gait events based on acceleration.

### BACKGROUND

Gait refers to the movement posture shown when walking, which is a continuous periodic movement. Affected by factors such as exercise habits, disability, disease, etc., different people generally have different gait patterns. The research and application of gait patterns are divided into two directions: a gait recognition and a gait analysis. The gait recognition is where researchers in the field of pattern recognition use gait patterns to identify pedestrians; the gait analysis is where researchers in the medical field use gait patterns for disease diagnosis. Both of these applications are based on the detection of gait periodic events. Therefore, the automatic detection of gait periodic events is a fundamental issue of artificial intelligence applications such as gait recognition and medical abnormal gait analysis.

At present, gait event detection methods are mainly divided into two categories: a wearable sensor-based gait event detection methods and a vision-based gait event detection method. The vision-based gait event detection party can directly detect gait events from the video data captured by a single or multiple cameras, without the cooperation of any other special sensors. Compared with wearable sensors, cameras are cheaper and more convenient to use. However, changes in lighting, perspective, and clothing make it more challenging to detect gait events from two-dimensional video data. Hand-crafted features such as edges and gradients are not discriminative, and they are more sensitive to lighting and perspective. A wearable sensor-based gait event detection method can accurately detect gait events by collecting motion data of the joints and segments of the human body. Because of its high accuracy, it has been widely used in the evaluation of abnormal gait in the medical field. Force plates and optical motion capture systems are the most common gait detection equipment, but they are expensive and require harsh application conditions. In addition, people also use some movable, lightweight, low-cost detection equipment, such as plantar pressure switches embedded in the soles, wearable accelerometers and gyroscopes for gait event detection, but most of them require a high degree of cooperation from the tested person. Therefore, it is of great significance to design a low-cost and accurate algorithm for gait event detection.

### SUMMARY

The technical problem to be solved by the disclosure is to provide the method and device for detecting gait events based on acceleration, which recognizes gait events by analyzing a three-axis acceleration data, improves the utilization rate of hardware resources, reduces the number of sensors, improve the accuracy of gait event detection, reduce the consumption of computing resources, and solve the bottleneck problems of expensive equipment required for gait event recognition based on wearable sensors, harsh application conditions, and the need for a high degree of cooperation from the subject.

The technical solutions to solve the above technical problems are as follows:

S1, obtaining a three-axis acceleration energy signal and a three-axis acceleration signal, and filtering and smoothing the three-axis acceleration energy signal and the three-axis acceleration signal, a three-axis acceleration includes an anteroposterior (AP) acceleration, a vertical (VT) acceleration and a medio-lateral (ML) acceleration:

S2, using a peak detection and a zero-crossing detection to screen points of local maximum and exceeding the peak threshold from processed vertical acceleration signal to form a point set ZC2;

S3, for any point ZCₖ in the point set ZC2, searching for the maximum value of the anteroposterior acceleration within a preset search window centered on the abscissa of the point ZCₖ, then a time corresponding to this value is the time when a heel strike, and a time corresponding to the nearest wave trough to the right of a value is the time when a toe off.

Further, in the method for detecting gait events based on acceleration, comprising filtering and smoothing the three-axis acceleration signal:
using three Gaussian filters with different σ to filter a three-axis acceleration energy signal e(i), then get three filtered signals e_{σ1} (i), e_{σ2}(i), e_{σ3}(i); and σ₁<σ₂<σ₃;

in order to retain most of the energy of the energy signal, and to suppress noise as much as possible, multiplying the three filtered signals e_{σ1} (i), e_{σ2}(i), and e_{σ3}(i) to obtain an output signal pₑ(i);

subtracting the filtered signal e_{σ3}(i) of the output signal pₑ(i) to obtain a signal sₑ(i);

The method for detecting gait events based on acceleration, comprising values of σ₁, σ₂, and σ₃ that are determined by steptime, and a value range of steptime is from Tmin to Tmax, Tmin is the minimum value of steptime, and Tmax is the maximum value of steptime. The values of σ of the three Gaussian filters are as follows: σ₁=Tmin/4, σ₂=(Tmin+Tmax)/4, σ₃=Tmax/4.

Further, in the method for detecting gait events based on acceleration, comprising filtering and smoothing the three-axis acceleration signal:

using two different a Gaussian filters to filter the vertical acceleration y(i) in the three-axis acceleration signal to obtain signals y_{σ4}(i) and y_{σ5}(i); σ₄<σ₅;

calculating a signal s_{y}(i), s_{y}(i)=y_{σ4}(i)−y_{σ5}(i).

Further, in the method for detecting gait events based on acceleration, using the peak detection and the zero-crossing detection to screen points of local maximum and exceeding the peak threshold from processed vertical acceleration signal to form a point set ZC2 comprises:

performing the peak detection and the zero-crossing detection on the signal s_{y}(i) to obtain a peak point set PK_{sy} and a zero-crossing point set ZC;

eliminating a zero-crossing point in the set ZC with an ordinate from positive to negative, and get a set ZC1; and

setting the peak threshold and filtering the zero-crossing points in the set ZC1: if an abscissa difference between a zero-crossing point ZCₖ in the set ZC1 and a nearest peak point on the right is less than a preset value and an amplitude of the nearest peak point is greater than the peak threshold, the zero-crossing point ZCₖ is extracted to form a set ZC2.

Further, in the method for detecting gait events based on acceleration, the peak threshold value is L times the maximum peak point amplitude of the signal sₑ(i) obtained after filtering and smoothing the three-axis acceleration energy signal, and the value range of L is from 0.45 to 0.65.

Further, the method for detecting gait events based on acceleration, comprising the following steps:

signal acquisition and preprocessing module, which is used to obtain;

the three-axis acceleration energy signal and the three-axis acceleration signal, and filter and smooth the three-axis acceleration energy signal and the three-axis acceleration signal, a three-axis acceleration includes the AP acceleration, the VT acceleration and the ML acceleration;

filter module, using the peak detection and the zero-crossing detection to screen points of local maximum and exceeding the peak threshold from processed vertical acceleration signal to form a point set ZC2;

positioning module, for any point ZCₖ in the point set ZC2, search for the maximum value of the forward and backward acceleration within a preset search window centered on the abscissa of the point ZCₖ, then the time corresponding to this value is the time when the heel strike (HS), and the time corresponding to the nearest wave trough to the right of the value is the time when the toe off (TO).

Further, an electronic device, comprising:

a memory, used to store computer software programs;

a processor is used to read and execute the computer software program stored in the memory to realize the detecting gait events based on acceleration method.

Further, a non-volatile computer-readable storage medium, wherein the storage medium stores a computer software programmer for implementing the detecting gait events based on acceleration method.

The beneficial effects of the disclosure are as follow: the disclosure only relies on a set of three-axis acceleration data to recognize gait events, which solves the bottleneck problems of expensive equipment required for gait event recognition based on wearable sensors, harsh application conditions, and high degree of cooperation from the subject, and improve the utilization of hardware resources, reduce the number of sensors, improve the accuracy of gait event detection, and reduce the consumption of computing resources.

### BRIEF DESCRIPTION OF DRAWINGS

FIG. 1 is a schematic diagram of a gait event in one step;

FIG. 2 is a schematic diagram of AP acceleration curve;

FIG. 3 is a schematic diagram of the three-axis acceleration direction;

FIG. 4 is a schematic diagram of the flow of the acceleration-based gait event detection method;

FIG. 5 is a schematic diagram of the structure of an acceleration-based gait event detection device.

### DETAILED DESCRIPTION OF THE EMBODIMENTS

The disclosure will be further described in detail below with reference to the embodiments and drawings. The exemplary embodiments of the present disclosure and the description thereof are only used to explain the present disclosure, not as a limitation of the present disclosure.

Basic calculation principles of running dynamic parameters:

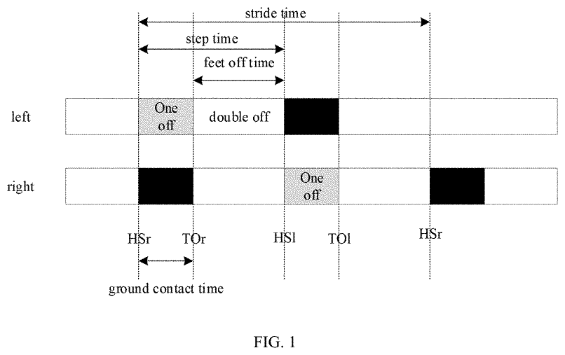

FIG. 1 shows the gait events involved in a stride during running. If the time of the corresponding gait event can be accurately located, some dynamic data such as the Step, Vertical Vibration (VVI), Ground Contact Time (GCT), step time and step frequency (step_fre) can be obtained through analysis:

GCT=Tor-HSr

VVI=½gt2,t=½(HS1-Tor)

Since a Tsample interval of two consecutive acceleration sensors is very short, the movement in this time period can be regarded as uniformly accelerated motion when calculating the step, from the initial moment, the initial velocity is v_{σ}, and the velocity after the ith sampling is vi=aAP×Tsample+vi-1, then the movement distance si=½(vi+vi-1) in the ith sampling interval Tsample, so it can be derived:

Step=Σᵢ₌₁ ^{(HSl-HSr)T}ₛₐₘₚₗₑₛSᵢ

The time interval between two consecutive HS is the step time (seconds):

steptime=HS(i)−HS(i−1)

Step frequency is the number of steps taken in one minute (60 seconds):

step_fre=60/steptime

Average step time of n steps (steptime_{average}):

$${steptime}_{average} = \frac{1}{n} {\sum }_{i = 1}^{n} steptime ( i )$$

Average n step frequency (step_fre_{average}):

step_fre_{average}=60/steptime_{average}

### Embodiment 1

The present embodiment provides the method for detecting a gait event based on acceleration. Define the identification criteria of HS and TO: in the AP acceleration signal, the relevant maximum peak is the time when the HS event occurs, and the local minimum peak in a small neighborhood after the HS event occurs is the time when the TO event occurs.

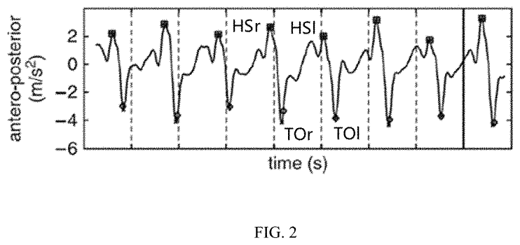

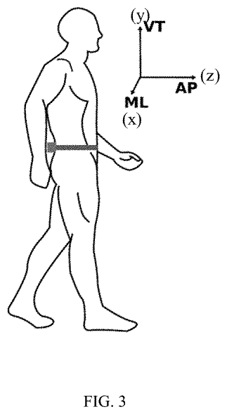

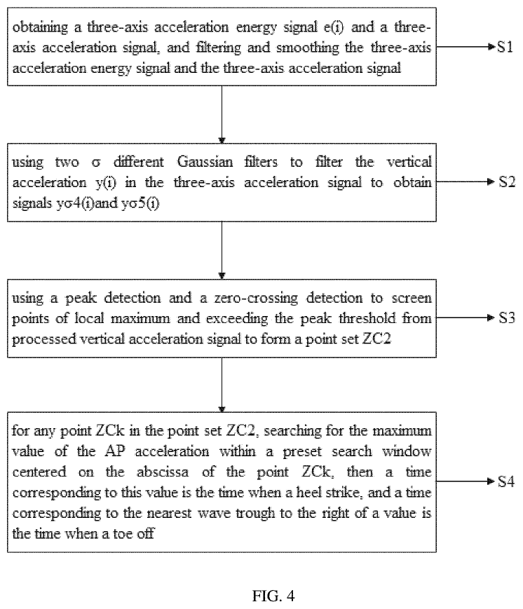

However, there are many local maximums and local minimums in the AP acceleration signal. As shown in FIG. 2, it is not easy to directly determine HS and TO, use the three-axis accelerometer to get the acceleration in the three directions of AP, VT, and ML, as shown in FIG. 3, the acceleration signal is filtered and smoothed, and after the local interference is eliminated, peak detection and zero-crossing detection are used to locate the time when the HS and TO events occur, specifically, the method, as shown in FIG. 4, includes the following steps:

S1: acquiring a three-axis acceleration energy signal e(i) and a three-axis acceleration signal at time i, and perform filtering and smoothing processing on the three-axis acceleration energy signal and the three-axis acceleration signal;

the three-axis acceleration including the AP acceleration x(i), the VT acceleration y(i), and the ML acceleration z(i); the sampling times of x(i), y(i), and z(i) are the same, and the abscissa is uniform, the three-axis acceleration energy signal in this example refers to the amplitude of the vector sum of the three-axis acceleration measured by the sensor, because the individual step lengths are different, the e(i) signal is filtered and smoothed by 3 Gaussian filters with different parameters to obtain 3 filtered signals e_{σ1} (i), e_{σ2}(i), e_{σ3}(i);

the values of the Gaussian filter parameters σ1, σ2, and σ3 are determined by the predetermined steptime range. The larger the σ, the stronger the smoothing effect produced by the Gaussian filter. The value range of steptime steptime is from Tmin toTmax, Tmin is the minimum value of steptime, and Tmax is the maximum value of steptime. The values of a of the three Gaussian filters are as follows: σ₁=Tmin/4, σ₂=(Tmin+Tmax)/4, σ₃=Tmax/4;

in order to retain most of the energy of the energy signal, suppress noise as much as possible, multiplying the three filtered signals e_{σ1}(i), e_{σ2}(i), and e_{σ3}(i) to obtain an output signal pₑ(i), and then subtracting the filtered signal e_{σ3}(i) from the output signal pₑ(i) to obtain the signal sₑ(i), which contains a lot of energy and is very close to HS.

S2, using two different a Gaussian filters to filter the vertical acceleration y(i) in the three-axis acceleration signal to obtain the signals y_{σ4}(i) and y_{σ5}(i); in this embodiment, σ₄=50 ms, σ₅=100 ms;

comparing y_{σ4}(i) with the anteroposterior acceleration signal z(i), it is found from experience that the HS event occurs between the local maximum value of y_{σ4}(i) and the previous local minimum value, the signal curves of y_{σ4}(i) and y_{σ5}(i) intersect at a point between the local maximum and the local minimum, using zero-crossing detection and subtracting the two signals to get S_{y}(i), S_{y}(i)=y_{σ4}(i)−y_{σ5}(i), to find the abscissa of the intersection of the two signals of y_{σ4}(i) and y_{σ5}(i),and then finding the intersection of S_{y}(i) and the y-axis.

S3, using the peak detection and the zero-crossing detection to screen points of local maximum and exceeding the peak threshold from processed vertical acceleration signal to form a point set ZC2;

performing the peak detection and the zero-crossing detection on the signal S_{y}(i) to obtain the peak point set PK_{sy} and the zero-crossing point set ZC;

eliminating the zero-crossing points with the ordinate from positive to negative in the set ZC, and get the set ZC1;

filtering out the small amplitude peaks caused by noise, the peak threshold is set to be L times the maximum peak point amplitude of the signal sₑ(i). The value of L is determined based on experience. The general value range is from 0.45 to 0.65. In this embodiment, the peak threshold is 0.51 times the amplitude of the maximum peak point, and the corresponding amplitude is 0.13;

detecting the abscissa difference between the zero-crossing point ZCₖ in the set ZC1 and the nearest peak point on the right in S_{y}(i), if the abscissa difference between the zero-crossing point ZCₖ in the set ZC1 and the nearest peak point pi on the right side |pi-ZCₖ|<TH and the amplitude of the peak point pi is greater than the peak threshold, then the zero-crossing point ZCₖ is extracted to form a set ZC2;

what needs to be explained is that TH is a preset threshold to determine a search range to ensure the completeness and accuracy of the gait event detection results. There are many zero-crossing points in the signal S_{y}(i), and the value of TH is related to the average value of the distance between adjacent zero-crossing points. For example, TH can be ⅓ of the average value of the distance.

S4, for any point ZCₖ in the point set ZC2, searching for the maximum value of the AP acceleration within the preset search window centered on the abscissa of the point ZCₖ, then the time corresponding to this value is the heel strike (HS) time. The moment corresponding to the right nearest wave trough is the TO moment;

the preset search window range here is (ZCₖ-TH, ZCₖ+TH);

locating the time of the HS and TO events, the following methods can be used to derive and calculate the running parameters:
1. Judging algorithm for left and right feet:

taking an abscissa of the first m (m is odd) peak points of the X axis to form an array [t1, t2, t3 . . . tm], and taking the abscissas of all the peak points of the Y axis to form an array [T1, T2, T3 . . . Tn], build m new arrays based on the above two arrays:

[T1-t1, T2-t1, T3-t1 . . . Tn-t1],

[T1-t2, T2-t2, T3-t2 . . . Tn-t2],

[T1-tm, T2-tm, T3-tm . . . Tn-tm];

taking an absolute value of all the elements of m arrays, and find a serial number of the smallest number in each array, according to the parity classification of the serial number, the number of odd serial numbers and the number of even serial numbers are counted, if the number of odd numbers is greater than the number of even numbers, the peak point with the odd number on the Y axis corresponds to the HSr; if the number of odd numbers is less than the number of even numbers, the peak point with an even number on the Y axis corresponds to the HSr;
2. Grounding contact balance algorithm;

the purpose of bottom-bottom balance detection is to detecting the stress on the feet of the runner and avoiding injury to the legs during running;

in an array composed of 2K consecutive ground contact times, the algorithm for ground contact balance is: the sum of odd numbered ground contact times divided by the sum of all ground contact times:

$$\begin{matrix}GCT\_odd = {\sum }_{i = 1}^{K} GCT ( 2 i - 1 ) \\ GCT\_all = {\sum }_{i = 1}^{2 K} GCT ( i ) \\ GCT\_balance = \frac{GCT\_odd}{GCT\_all}\end{matrix}$$

### Embodiment 2

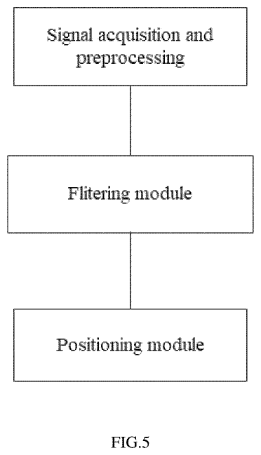

The embodiment of objective provides a method for detecting gait events based on acceleration, as shown in FIG. 5, including:

signal acquisition and preprocessing module, which is used to acquire the three-axis acceleration energy signal and the three-axis acceleration signal, and perform filtering and smoothing processing on the three-axis acceleration energy signal and the three-axis acceleration signal; the three-axis acceleration includes the AP acceleration, the VT acceleration and the ML acceleration;

filter module, using the peak detection and the zero-crossing detection to screen points of local maximum and exceeding the peak threshold from processed vertical acceleration signal to form a point set ZC2;

positioning module, for any point ZCₖ in the point set ZC2, searching for the maximum value of the forward and backward acceleration within a preset search window centered on the abscissa of the point ZCₖ, then the time corresponding to this value is the time HS, and the time corresponding to the nearest wave trough to the right of the value is the TO.

### Embodiment 3

The embodiment of the disclosure provides an electronic device including the memory and the processor; the memory's inner store including a computer software program; a processor reads and executes the computer software program stored in the memory to realize an acceleration-based gait Event detection method, realizing an acceleration-based gait event detection method, comprising the following steps:

S1, obtaining the three-axis acceleration energy signal and the three-axis acceleration signal, and filtering and smoothing the three-axis acceleration energy signal and the three-axis acceleration signal, the three-axis acceleration includes the anteroposterior (AP) acceleration, the vertical (VT) acceleration and the medio-lateral (ML) acceleration;

S2, using the two Gaussian filters with different a to filter the vertical acceleration y(i) in the three-axis acceleration signal to obtain signals y_{σ4}(i) and y_{σ5}(i);

S3, using the peak detection and the zero-crossing detection to screen points of local maximum and exceeding the peak threshold from processed vertical acceleration signal to form a point set ZC2;

S4, for any point ZCₖ in the point set ZC2, searching for the maximum value of the AP acceleration within the preset search window centered on the abscissa of the point ZCₖ, then the time corresponding to this value is the HS time, the moment corresponding to the right nearest wave trough is the TO;

what should be noted is that, in the foregoing embodiments, the description of each embodiment has its own focus, and for parts that are not described in detail in an embodiment, reference may be made to related descriptions of other embodiments.

People who skilled in the art should understand that the embodiments of the present disclosure can be provided as a method, a system, or a computer program product, therefore, the objection may adopt the form of a complete hardware embodiment, a complete software embodiment, or an embodiment combining software and hardware, moreover, the objection may adopt the form of a computer program product implemented on one or more computer-usable storage media (including but not limited to a disk storage, a CD-ROM, an optical storage, etc.) containing computer-usable program codes.

The disclosure is described with reference to flowcharts and/or block diagrams of methods, devices (systems), and computer program products according to embodiments of the present disclosure. It should be understood that each process and/or block in the flowchart and/or block diagram, and the combination of processes and/or blocks in the flowchart and/or block diagram can be implemented by computer program instructions. These computer program instructions can be provided to the processor of a general-purpose computer, a special-purpose computer, an embedded computer, or other programmable data processing equipment to generate a machine, so that the instructions executed by the processor of the computer or other programmable data processing equipment are generated for A device that implements the functions specified in one process or multiple processes in the flowchart and/or one block or multiple blocks in the block diagram.

The computer program instructions can also be stored in a computer-readable memory that can boot a computer or other programmable data processing equipment to work in a specific manner, so that the instructions stored in the computer-readable memory produce an article of manufacture including the instruction device. The device implements the functions specified in one process or multiple processes in the flowchart and/or one block or multiple blocks in the block diagram.

The computer program instructions can also be loaded on a computer or other programmable data processing equipment, so that a series of operation steps are executed on the computer or other programmable equipment to produce computer-implemented processing, so as to execute on the computer or other programmable equipment. The instructions provide steps for implementing functions specified in a flow or multiple flows in the flowchart and/or a block or multiple blocks in the block diagram.

Although the preferred embodiments of the objection have been described, those skilled in the art can make additional changes and modifications to these embodiments once they learn the basic creative concept. Therefore, the appended claims are intended to be interpreted as including the preferred embodiments and all changes and modifications falling within the scope of the present disclosure.

Obviously, people who skilled in the art can make various changes and modifications to the present disclosure without departing from the spirit and scope of the present disclosure. In this way, if these modifications and variations of the present disclosure fall within the scope of the claims of the present disclosure and equivalent technologies, the present disclosure is also intended to include these modifications and variations.

What is claimed is:

1. A method for detecting gait events based on acceleration, comprising the following steps:
S1, obtaining a three-axis acceleration energy signal and a three-axis acceleration signal, and filtering and smoothing the three-axis acceleration energy signal and the three-axis acceleration signal, a three-axis acceleration includes an anteroposterior acceleration, a vertical acceleration and a medio-lateral acceleration;
S2, using a peak detection and a zero-crossing detection to screen points of local maximum and exceeding the peak threshold from processed vertical acceleration signal to form a point set ZC2; and
S3, for any point ZCₖ in the point set ZC2, searching for the maximum value of the AP acceleration within a preset search window centered on the abscissa of the point ZCₖ, then a time corresponding to this value is the time when a heel strike, and a time corresponding to the nearest wave trough to the right of a value is the time when a toe off.

2. The method for detecting gait events based on acceleration according to claim 1, wherein filtering and smoothing the three-axis acceleration energy signal comprise:
using three Gaussian filters with different standard deviation σ to filter a three-axis acceleration energy signal e(i), then get three filtered signals e_{σ1} (i), e_{σ2}(i), e_{σ3}(i); and σ1<σ2<σ3;
multiplying the three filtered signals eσ1 (i), e_{σ2}(i), and e_{σ3}(i) to obtain an output signal pₑ(i); and
subtracting the filtered signal e_{σ3}(i) of the output signal pₑ(i) to obtain a signal sₑ(i).

3. The method for detecting gait events based on acceleration according to claim 1, comprising values of σ1, σ2, and σ3 that are determined by steptime, and a value range of steptime is from Tmin to Tmax.

4. The method for detecting gait events based on acceleration according to claim 1, wherein: σ₁=Tmin/4, σ₂=(Tmin+Tmax)/4, σ₃=Tmax/4.

5. The method for detecting gait events based on acceleration according to claim 1, comprising filtering and smoothing the three-axis acceleration signal:
using two σ different Gaussian filters to filter the vertical acceleration y(i) in the three-axis acceleration signal to obtain signals y_{σ4}(i) and y_{σ5}(i); σ₄<σ₅; and
calculating a signal S_{y}(i), s_{y}(i)=y_{σ4} (i)y_{σ5}(i).

6. The method for detecting gait events based on acceleration according to claim 1, comprising using the peak detection and the zero-crossing detection to screen the points of local maximum and exceeding the peak threshold from processed vertical acceleration signal to form a point set ZC2:
performing the peak detection and the zero-crossing detection on the signal S_{y}(i) to obtain a peak point set PK_{sy} and a zero-crossing point set ZC;
eliminating a zero-crossing point in the set ZC with an ordinate from positive to negative, and get a set ZC1; and
setting the peak threshold and filtering the zero-crossing points in the set ZC1: If an abscissa difference between a zero-crossing point ZCₖ in the set ZC1 and a nearest peak point on the right is less than a preset value and an amplitude of the nearest peak point is greater than the peak threshold, the zero-crossing point ZCₖ is extracted to form a set ZC2.

7. The method for detecting gait events based on acceleration according to claim 1, wherein the peak threshold value is L times a maximum peak point amplitude of the signal sₑ(i) obtained after filtering and smoothing the three-axis acceleration energy signal, and the value range of L is from 0.45 to 0.65.

8. A device to detect gait events based on acceleration, wherein:
signal acquisition and preprocessing module, obtaining the three-axis acceleration energy signal and the three-axis acceleration signal, and filter and smooth the three-axis acceleration energy signal and the three-axis acceleration signal, a three-axis acceleration includes the forward and backward acceleration, the vertical acceleration and the lateral acceleration;
filter module, using the peak detection and the zero-crossing detection to screen points that are locally largest and exceed the peak threshold in the processed a vertical acceleration signal to form the point set ZC2; and
positioning module, for any point ZCₖ in the point set ZC2, search for the maximum value of the anteroposterior acceleration within a preset search window centered on the abscissa of the point ZCₖ, then the time corresponding to this value is the time HS, and the time corresponding to the nearest wave trough to the right of the value is the TO.

9. An electronic device, wherein:
a memory used to store computer software programs; and
a processor used to read and execute the computer software program stored in the memory to realize the detecting gait events based on acceleration method of claim 1.

10. A non-volatile computer-readable storage medium, wherein the storage medium stores a computer software programmer for implementing the detecting gait events based on acceleration method of claim 1.
